(19) United States
(12) Patent Application Publication
(10) Pub. No.: US 2020/0057039 A1
(43) Pub. Date: Feb. 20, 2020

(54) DEVICE AND METHOD FOR DETECTING GAS

(71) Applicant: ETH Zurich, Zurich (CH)

(72) Inventors: Andreas T. Güntner, Zurich (CH); Sebastian Abegg, Wilen (CH); Karsten Wegner, Zurich (CH); Sotiris Pratsinis, Zurich (CH)

(21) Appl. No.: 16/345,781

(22) PCT Filed: Nov. 1, 2017
(86) PCT No.: PCT/EP2017/077979

§ 371 Date: Apr. 29, 2019

(30) Foreign Application Priority Data
Nov. 1, 2016 (CH) 01459/16

(51) Int. Cl. G01N 33/00

(52) U.S. Cl. CPC G01N 33/0014

(57) ABSTRACT

An analyzer for detection of a target compound in a complex gas mixture in the gas phase includes a detector for detecting the target compound and further has a sensing cavity. The detector is arranged in the sensing cavity. The analyzer also has a separate first membrane that is equipped to close the cavity and simplifies the composition of the complex gas mixture into a first gas mixture, wherein the first gas mixture includes the first target compound and wherein the first membrane is equipped to let the first gas mixture traverse through the first membrane into the sensing cavity. The detector is equipped to detect the first target compound.

The invention relates to the field of gas detection. It relates to a gas analyser for detection of gaseous target compound in a complex gas mixture and a method for gas detection as described in the preamble of the independent claims.

Modern chemical gas detectors (e.g. semiconductive metal oxides (ACS Sens. 2016, 1, 528-535) or carbon nanotubes (Nanotechnology 2010, 21, 185501)) can detect analytes at a parts-per-billion (ppb) level. However, a major challenge remains their lack of selectivity to the target compound, a necessary requirement to detect a compound in a complex gas mixture. Zeolitic materials have already been applied to tackle the selectivity issue of gas sensors. So far, these zeolitic materials were either used as packed beds (Hugon Sens. Actuators B 2000, GB2068561) or as coatings on the sensor material (Tian ACS Sensors 2015, JPS6390752). While packed beds consist of zeolitic particles and are positioned upstream the sensor, the coating is directly placed on the sensing material and thus coupled mechanically and thermally. State-of-the-art packed beds and coatings suffer from significant drawbacks: Packed beds only remove molecules smaller than the pore size of the zeolitic material, while larger molecules may interact with the sensor. Therefore, simplifying the gas mixture by a physical size cutoff, i.e. hindering molecules larger than the pore size from interaction with the sensor, is not possible. Furthermore, the zeolitic particles saturate and need regeneration. Zeolitic coatings should hinder larger molecules than the pore size to access the sensor surface. However, since they are thermally coupled to the sensor that is typically heated to 200-500° C., catalytic reactions can occur on the surface of the zeolitic coating and/or inside the zeolitic coating, altering the gas composition by generation of new undesired compounds that interfere with the sensor, deteriorating the separation efficiency and thus the sensor selectivity. Target molecules may be catalytically converted as well and are not detectable anymore. Moreover, the application of the coating may negatively alter the sensing material during sensor fabrication.

Detector systems for detection of at species in vapour are known e.g. form U.S. Pat. No. 4,745,986, where an immobilized liquid membrane (ILM) with big liquid filled pores is combined with a sensor to detect the presence of tobacco smoke. JP 2010025718 shows an expiration measurement device which a permeable membrane.

It is therefore an object of the invention to create an improved analyser for detection of a gaseous target compound from/in a complex gas mixture and a method for detection of a target compound in a complex gas mixture, e.g. in breath analysis and air quality monitoring.

These objects are achieved by an analyser and a method according to the independent claims.

An analyser for detection of a target compound in respectively from a complex gas mixture in the gas phase is provided. The analyser comprises a detector for detecting the target compound and further comprises a sensing cavity. The detector is arranged in the sensing cavity. The analyser further comprises a separate first membrane, wherein the first membrane is equipped to close the cavity. Furthermore, the first membrane is equipped for simplifying the composition of the complex gas mixture into a first gas mixture, wherein the first gas mixture comprises the first target compound and wherein the first membrane is equipped to let the first gas mixture traverse through the first membrane into the sensing cavity. The detector is equipped to detect the first target compound. In other words: The membrane is equipped to allow for the first gas mixture to traverse through the first membrane into the cavity. The complex gas mixture comprises more than two components. For example air is a complex gas mixture with main components nitrogen and oxygen and further compounds at a trace level, like argon and carbon dioxide. The complex gas mixture is in the gas phase and is not dissolved in a liquid or fluid.

The simplification of the composition of the complex gas mixture implies that the first gas mixture comprises less components respectively compounds than the complex gas mixture or respectively that relative concentration of the target compound in the first gas mixture is increased with respect to the “original” complex gas mixture outside the sensing cavity. At least the first target compound is capable to traverse through the first membrane and other compounds are held back and are essentially prevented from entering the sensing cavity. Essentially mainly the target compound is capable of traversing through the membrane. Accordingly, the simplification of the composition of the complex gas mixture results in the separation of the complex gas mixture into a first gas mixture and a remaining gas mixture. The remaining gas mixture could be further analysed if needed. The first gas mixture comprises an enriched relative concentration of the target compound as compared to the complex gas mixture. Consequently the concentration of components/compounds not being the target compound is lowered/reduced in the first gas mixture as compared to the their concentration in the complex gas mixture. Accordingly they do not have or a reduced influence on the detection of the target compound. The remaining gas mixture comprises a lower relative concentration of the target compound compared with the complex gas mixture. The first gas mixture may be analysed directly respectively the target compound may be detected directly by the detector or be further simplified.

The membrane is equipped to close the sensing cavity. The cavity can be formed by a piece of a wall of a container and/or of a surface and the membrane is capable to build a self-contained volume that is at least partially separated from the complex gas mixture. The self-contained volume comprises the first gas mixture being different from the complex gas mixture present outside of the cavity.

Detecting the target compound comprises at least one of a sensing respectively identification of the target compound and a quantifying of the concentration of the target compound. This means the term detection may refer to the detection/sensing of the presence of the target compound as well as to the quantification of its (target compounds) concentration.

The term separated with respect to the membrane means that the membrane is spatially separated from the detector. The means that essentially no physical contact between the detector and the membrane occurs. Furthermore, the membrane is separate from the sensing cavity and accordingly they are not provided as a single piece.

The membrane can be a single piece membrane. The membrane can be equipped for essentially only letting the target compound traverse into the cavity. A compound not being the target compound might be called non-target compound. The membrane can be equipped for letting a non-target compound traverse into the sensing cavity, wherein the non-target compound essentially does not interfere with the detection of the target compound in the cavity by the detector. In contrast to the packed bed zeolitic material the membrane enables a cut-off of molecules/components larger than the pore size. Furthermore, in contrast to the zeolitic coating the membrane is mechanically and thermally decoupled form the detector avoiding catalytic reactions influencing the composition of the gas. Additionally, the application of the zeolitic coating on top of the sensor influence does the sensor properties.

The membrane can be capable of performing a chemical separation and a physical separation of the components respectively composition of the complex gas mixture. The physical separation of the complex gas mixture is based on the size respectively geometry of the pores of the membrane. The physical separation can be view as a size separation, in particular with a “cut-off” size of molecules capable to traverse through the membrane. The chemical separation of the complex gas mixture is based on the chemical respectively electrostatic (i.e. Van der Waals forces) interaction between the membrane and the components of the gas mixture. Due the physical and chemical separation in parallel an effective separation is achieved.

The membrane can comprise a pore size with a diameter respectively cross section of maximally 10 nm, in particular maximally 2 nm, in particular maximally 1 nm, in particular 0.6 nm.

The physical separation can be enabled by the size of the pores of the membrane. The size of the pores of the membrane can be smaller than the molecules which are physically separated. Mesoporous and macroporous materials as well as amorphous materials are not suitable for a physical separation of volatile molecules (e.g. NH3; Acetone, Formaldehyde) because their pore size is too large.

A chemical separation can be influenced by the adsorption properties of the material with respect to a specific molecule respectively a specific group of molecules. For example hydrophilic materials can be suitable for the separation of hydrophilic and hydrophobic components. Such materials can achieve a high selectivity. Nevertheless, a solely adsorption membrane cannot provide a physical separation of the components of the complex gas mixture.

The membrane can be arranged in a dead-end geometry, where the gas mixture respectively a flow of the gas mixture is directed essentially normal to the membrane surface. Alternatively, the membrane can be arranged in a cross-flow or tangential flow arrangement, where the feed flow of the gas mixture is essentially tangential to the surface of the membrane.

The analyser may be equipped for medical and biological fluid analysis, in particular breath analysis, analysis of skin emissions, headspace analysis of fluids. The analyser may be a breath detector, skin analyser, headspace analyser for fluids. Furthermore, the analyser may be equipped for food processing control, food quality assessment monitoring of agricultural processes and products, monitoring and control of chemical processes, indoor air analysis, environmental analysis, detection of explosives and other hazardous compounds, exhaust emission monitoring and control and/or air quality analysis. The analyser may be a food processing analyser, food quality analyser and/or an air quality analyser. Accordingly, the detection of a target compound originating from a medical or biological fluid for example in breath, from skin emissions, during food processing, in food or air is enabled. In the headspace analysis and respectively other analyses of fluids only the gas phase above the fluid is analysed, accordingly it might be called a (headspace) analysis of fluids in the gas phase.

The analyser can comprise an inlet and an outlet. The inlet is arranged such that the complex gas mixture is guided onto the membrane closing the sensing cavity. The first membrane may be arrangeable between the inlet and the outlet. The outlet may be arranged in an axial and/or non-axial orientation with respect to the inlet. Accordingly, the interaction between the complex gas mixture and the membrane is promoted and the simplification of the complex gas mixture is assisted.

The inlet may comprise a mouthpiece though which a patient can breathe. Hence the guiding of the complex gas mixture, in particular the breath, towards the membrane is enhanced and detection of the target compound might be promoted. The patient might be a human or an animal, while breathing means inhaling and/or exhaling. In particular the patient can exhale through the mouth piece.

It might be possible that the complex gas mixture is actively pumped towards the membrane. It might also be that the complex gas mixture is passively diffusing towards the membrane. Furthermore it might be possible that the complex gas mixture is conveyed towards the membrane due to a pressure difference.

It is possible, that the complex gas mixture is guided towards the membrane with a pump and/or a fan. Thereby, the complex gas mixture might be guided from a sampler (collection of the complex gas mixture) via the pump to the sensing cavity with the detector.

The membrane might be equipped to block the direct flow of the complex gas mixture into the sensing cavity. This enhances a selective detection of the simplified gas composition of the first gas mixture. Such a blocking of the direct flow might also be called closing of the sensing cavity.

The sensing cavity may comprises a detector housing with an opening, wherein the membrane, in particular the first membrane and/or a second membrane, is arrangeable in the opening. The detector can be arranged in the detector housing, wherein the detector housing substantially surrounds the detector. Naturally the housing can have an opening though which the detector can be introduced and/or though which the gas mixture can transverse.

The cavity may also comprise a base part, wherein the membrane, in particular the first membrane and/or a second membrane, is fastenable to the base part. In a fastened state membrane builds the sensing cavity together with the base part. In particular, the membrane can be pulled over the detector and builds the sensing cavity together with the base part. The membrane can be tube shaped wherein the sensing cavity is arranged inside the tube shaped membrane. The tube shaped membrane can be closed by the base part. The base part might be essentially flat. The base part might comprise a rim. The rim can be equipped for fastening the membrane to the base part. The rim and the membrane may each comprise a fastening means that enables the secure fastening of the membrane to the base part. The fastening means may comprise a flange. It is to be noted, that a space, i.e. a cavity, is arranged between the detector and the membrane in the fastened state. Thereby the space can between the membrane and the detector can be hollow.

The analyser can further comprise a separate second membrane. The first membrane is interchangeable with the second membrane. The second membrane can be equipped to close the sensing cavity sensing cavity. The second membrane can be equipped for simplifying the composition of the complex gas mixture into a second gas mixture, in particular for selectively simplifying the composition of the complex gas mixture into a second gas mixture. The second gas mixture comprises a second target compound. The second membrane is equipped to let the second gas mixture traverse through the second membrane into the sensing cavity. The detector is equipped to detect the second target compound. This can enable a modular analyser of several target compounds by exchange of the second membrane.

The analyser can be called modular analyser as the membrane can be interchanged in a modular manner.

The second target compound can be different from or identical to the first target compound. In the case of identical target compounds of the first and the second membrane the exchange respectively interchanging of the membranes leads to a reparation, restoration respectively renewal of the gas analyser.

It might also be possible, that the detector is exchanged for detecting the second target compound, wherein the first membrane remains in the sensing cavity. In such a case the first membrane is equipped to let the second gas mixture traverse through the first membrane into the sensing cavity. Accordingly, the second gas mixture may comprise the first and the second target compound, which are detected by the exchangeable detector.

The first gas mixture and the second gas mixture can have a different composition. This improves a modular detection of different components from the same complex gas mixture.

In other embodiments the first gas mixture and the second gas mixture can be equal to each other. This improves the life time of the analyser as the membrane can be exchanged without compromising the selective detection of a target compound.

The analyser can comprise at least a first sensing cavity and a second sensing cavity. The first membrane is equipped to close of the first sensing cavity and the second membrane is equipped to close the second sensing cavity. In the first sensing cavity a first detector is arranged. The first detector is equipped for detecting a first target compound. In the second sensing cavity a second detector is arranged. The second detector is equipped for detecting a second target compound. This enables a detection of at least two target compounds with the same analyser. Accordingly, the examinations costs and/or analysis time might be reduced due to the simultaneous and independent detection of the complex gas mixture with respect to different target compounds.

As already indicated above, the first gas mixture can comprise a first target compound as a component of the first gas mixture. Nevertheless, the first gas mixture can comprise in addition to the first target compound at least one further component, which might be detected with an additional detector in the same sensing cavity or in a different sensing cavity. This enables for a parallel detection of several compounds with the same analyser.

The first sensing cavity and the second sensing cavity can be arranged in a parallel manner and/or in a serial manner with respect to each other. Examples for such a parallel or serial arrangement of the cavities are described below. As a matter of course, the analyser can also comprise an array of sensing cavities with a number of membranes arranged in the individual cavities leading to an array like analysis of the complex gas mixture.

The first membrane and the second membrane can be equipped to close the sensing cavity. The sensing cavity can be the same sensing cavity and therefore the same cavity is closed by two membranes. This improves the simplification of the complex gas mixture as the first gas mixture traversing through the first membrane additionally has to traverse through the second membrane respectively the second gas mixture traversing through the second membrane additionally has to traverse through the first membrane. The combined effect of both membranes leads to a combined simplification of the complex gas mixture and/or to a selective detection of a target compound from a complex gas mixture. Furthermore, the first membrane and the second membrane may have the same properties enabling an improved selectivity of the separation and/or an improved throughput though the analyser. It is possible that the first membrane and the second membrane are arranged in a way that they contact each other or that a space is established between the membranes. For example it is possible that the membranes are applied directly on top of each other in a layered manner.

In the following several possibilities for combining membranes, sensing cavities and detectors for detection of a certain target compound or several target compounds according to the invention are listed:
several membranes might close the same cavity;
several detectors might be arranged in a single sensing cavity for parallel detection of various target compounds;
a single membrane closes a sensing cavity with one detector;
several membranes close a single cavity with one detector;
several membranes close a single cavity with several detectors arranged in the cavity;
all combinations of the above mentioned possibilities, for example several cavities with different detectors and membranes.

In particular the analyser can be equipped with at least one membrane, at least two membranes, at least three membranes, at least four membranes or more membranes.

The analyser can be equipped for selective detection of the concentration of the target compound in the complex gas mixture. Furthermore, the detector is equipped for detecting the concentration of the target compound. This improves the quantitative analysis of the target compound in the complex gas mixture. Such a quantitative analysis might be advantageous for the exact analysis of the gas mixture.

The analyser can be equipped for detection of target compound in the gas phase at a trace level. In particular the analyser can be equipped for detection of the target compound in a ppb range.

The term trace level means that the target compound is only present in a trace amount in the sample of the gas mixture. The target compound is only present in a minor concentration in the complex gas mixture. The term at a trace level could also mean, that minor changes in the concentration of the target compound can be determined, wherein the target compound may also be one of the main components of the complex gas mixture. The detected trace concentration or the trace chance of the concentration of the target compound in the gas mixture might be in the ppm and/or ppb range. ppm means part per million and is a measure for the concentration of a target compound, wherein 1 ppm means that 1 target molecule is present compared to 1 million (10⁺⁶) molecules of the whole gas mixture. Correspondingly, ppb means part per billion and 1 ppm equals to 1000 ppb. Therefore, 1 ppb represents 1 target molecule compared to 1 billion (10⁺⁹) molecules of the whole gas mixture.

The first membrane and/or the second membrane can be equipped for closing the cavity. A transition between the first and/or second membrane and the sensing cavity can be sealed, in particular the transition can be sealed by a gasket, a sealing, an 0-ring, a flange and/or an adhesive respectively a glue. For example the membrane can be arranged in a hollow, screw shaped insert and can be screwed against an O-ring arranged in the opening of the sensing cavity.

The membrane can be arranged in a removable manner at the sensing cavity. In a further example the membrane can be permanently fixed at the cavity, for example with an adhesive.

The first membrane respectively the second membrane can comprise a microporous material, with a pore size comparable to the size of the target compound. In particular the membrane can be a zeolite, a MOF (metal organic frameworks), a ZIF (zeolite imidazolate framework) and/or a CMS (carbon molecular sieve), in particular a MFI/alumina membrane. In particular the membrane can be a coin type zeolitic MFI layer on an Al₂O₃ support. MFI (Mobile five) is a type of a zeolitic crystal structure that comprises a three dimensional channel system. For example ZSM-5 (Zeolite Socony Mobil-5; Zeolite Socony Mobil-five) is an aluminosilicate zeolite with MFI crystal structure. ZIF is a subclass of MOF.

It is possible that the membrane comprises a layered structure with different materials. It is also possible that a layer of microporous material is equipped with a water-repellent respectively hydrophobic layer.

The first membrane respectively the second membrane can comprise a support element. In particular the support can be directly connected to the membrane. The support can comprise a ceramic material, a metal, a glass, steel and/or a polymer. Such a support respectively support element can provide additional stability to the membrane.

The support element can comprise a certain permeability with respect to the gas mixture.

The support can be equipped to influence the selectivity of gas detection. For example, in case a zeolite layer has a thickness of approximately 5 micrometer the support provides mechanical stability to the membrane. Nevertheless, the support contributes to the selectivity as the support is not totally inert. Accordingly, certain gases can be absorbed by the support material. The choice of the support may influence the separation properties of the membrane. The support may comprise the alpha phase of alumina (Al2O3) and/or stainless steel.

The membrane can be directly fastened to the support with a hydrothermal process. The membrane can be fastened to the support by chemical bonding and/or physical bonding (for example by a fastening means).

The detector can be at least one of a chemoresistive detector, a mass spectrometer, and an optical system. The detector can be selected from a group comprising
a doped SnO2 detector, in particular doped with Pt, Pd, Si, Ti;
a WO3 detector, in particular a Si-doped WO3 detector, in particular a epsilon phase of Si-doped WO3 detector;
a MoO3 detector, in particular a Si-doped MoO3 detector, in particular an alpha phase of Si-doped MoO3 detector;
a ZnO detector, in particular a Ti-doped ZnO;
a CuBr detector and
a CuO—SnO2 heterojunction.

In case several detectors are arranged in the analyser several selections can be performed.

The target compound can be a sulfuric compound in particular hydrogen sulphide and/or sulfur dioxide; a ketone, in particular acetone; a hydrocarbon, in particular isoprene; an aldehyde, in particular formaldehyde; an pnictogen hydride, in particular ammonia; an acid, in particular acetic acid; an alcohol, in particular methanol and/or ethanol; and/or hydrogen.

The analyser might be suitable for use in medical and biological fluid analysis; for use in breath analysis, in particular in lung cancer detection from exhaled breath, and/or for use in air quality analysis, in particular air quality monitoring of target compound released from indoor sources. A target compound released from indoor sources may originate from wood-based products and/or combusted biomass. Further examples are disclosed throughout the text.

The analyser can be used for medical and biological fluid analysis; for breath analysis, for analysis of skin emissions, for headspace analysis of fluids, for air quality analysis, for processing control, for food quality assessment and/or for air quality analysis; for monitoring and controlling agricultural and/or chemical processes and products; and/or for environmental analysis or monitoring and controlling exhaust emission; and/or for detection of explosives and other hazardous compounds.

A method for detecting a target compound in a complex gas mixture 6 comprises the step of:
providing an analyser as described above;
letting the complex gas mixture interact with the first membrane respectively a second membrane,

wherein the first gas mixture traverses through the membrane into the sensing cavity. The detector detects the target compound.

The membrane can enable a distinct molecular size cut off and a separation of the complex gas mixture. That way, compounds/molecules larger than the pore size cannot permeate through the membrane and thus, the complexity of the gas mixture can be reduced.

In the analyser the gas pressure inside the cavity is essentially similar to the gas pressure outside the sensing cavity. This means that the pressure difference between the inside and the outside of the sensing cavity can be very small. The pressure difference can be in the range of mbar. The inside of the cavity is accommodating the detector.

The analyser can comprise a zeolitic membrane, being the first and/or the second membrane, wherein the zeolitic membrane enhances the selectivity of the sensor.

The subject matter of the invention will be explained in more detail in the following text with reference to exemplary embodiments which are illustrated in the attached drawings, in which:

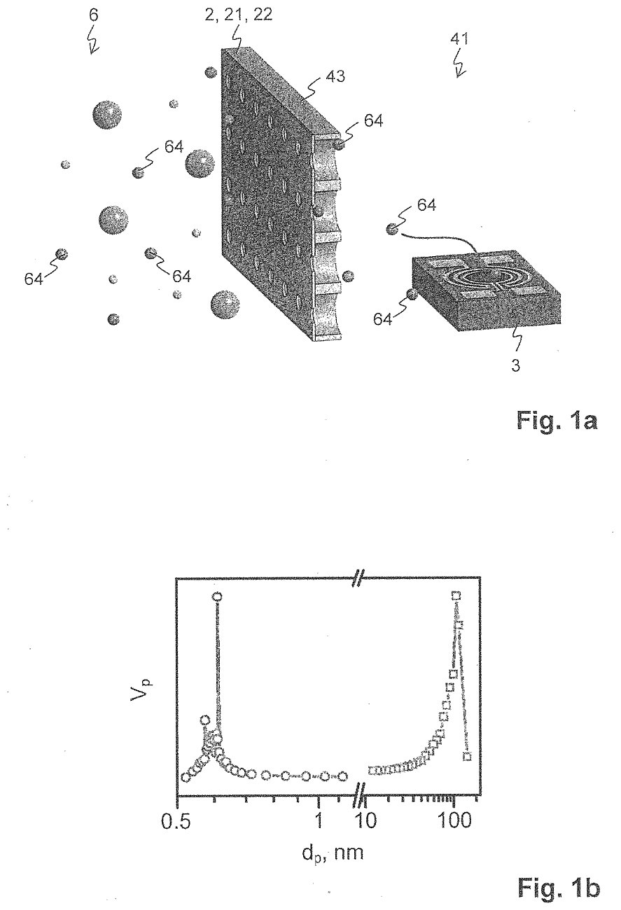

FIG. 1a Gas analyser 1, as membrane 2,21,22—detector 3 system;

FIG. 1b pore size of the membrane 2,21,22 and a support element;

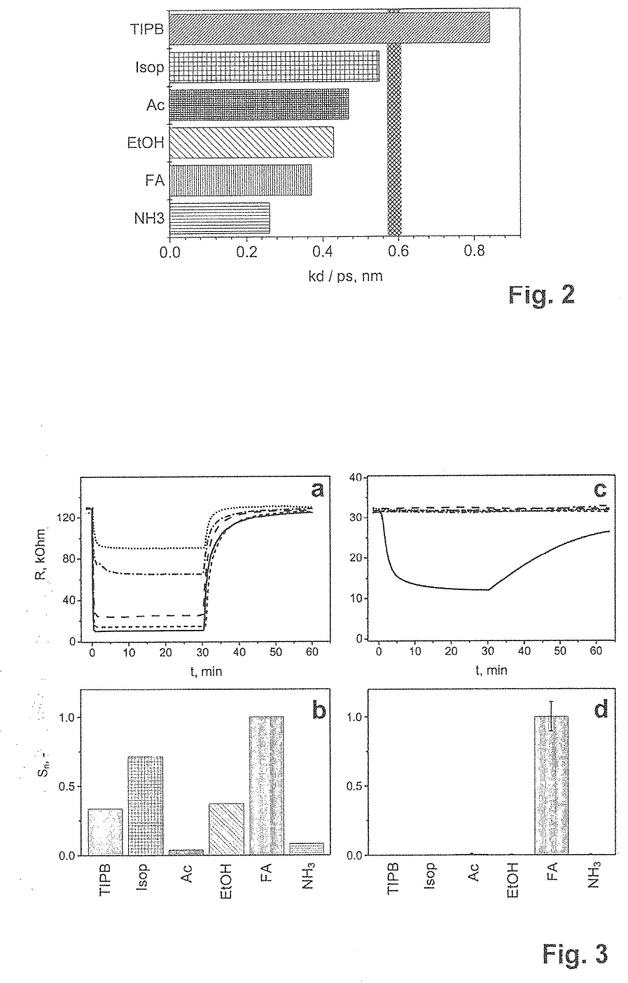

FIG. 2 target compounds 64 with their kinetic diameter in comparison to the determined MFI pore size (kd);

FIG. 3 Film resistance (R) and normalized response Sₙ of a detector 3 with and without a membrane 2 exposure to 1 ppm of acetone, NH₃, ethanol, isoprene and formaldehyde;

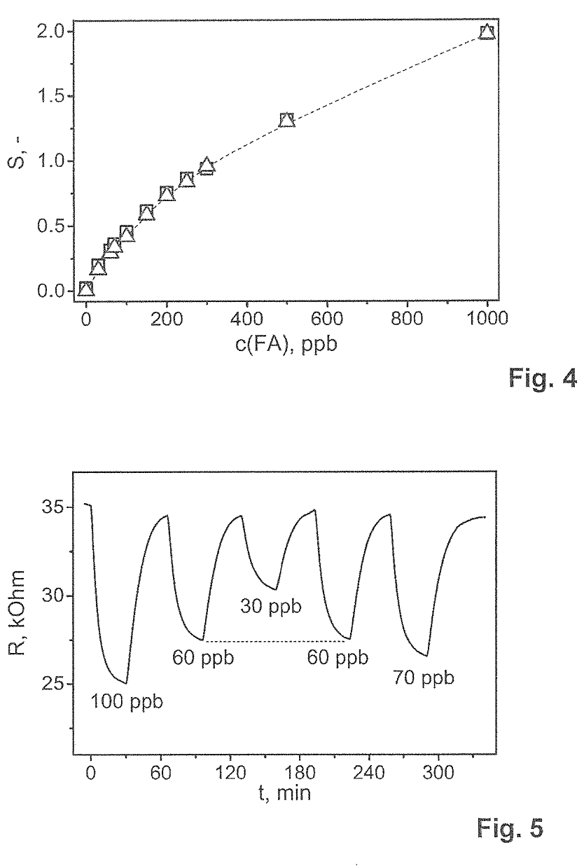

FIG. 4 detector 3 resistance of the membrane 2,21,22—detector 3 system upon exposure to 100, 70, 60 and 30 ppb of formaldehyde at 50% RH

FIG. 5 detector 3 calibration curve for formaldehyde in the range of 0-1000 ppb at 400° C. and 50% RH.

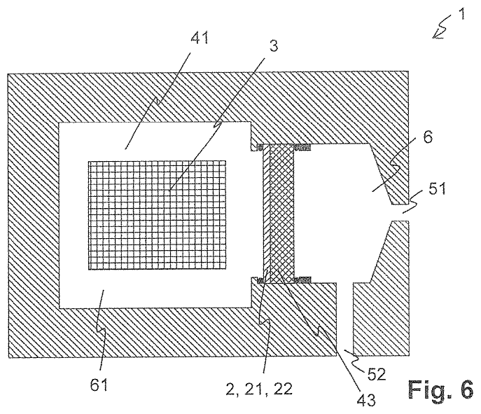

FIG. 6 sketch of an analyser 1

The reference symbols used in the drawings, and their meanings, are listed in summary form in the list of reference symbols. In principle, identical parts are provided with the same reference symbols in the figures.

### EXAMPLE

A MFI/alumina membrane enables a non-specific Pd-dopedSnO2 detector to selectively detect formaldehyde (FA).

Membrane Fabrication

A MFI precursor solution is prepared as follows: 1.4 g sodium hydroxide (97%, Sigma-Aldrich) are dissolved in 100 ml tetrapropylammonium hydroxide (1 M TPA(OH) in H₂O, Sigma-Aldrich) in a closed Teflon flask at room temperature. 20 g of fumed silica (Aerosil 200, Evonik) is added at ˜85° C. and dissolved under vigorous stirring and reflux. 3.2 mL of deionized water is added to the clear solution followed by a subsequent heating step to 105° C. for 15 min. The solution is cooled down within 45 min and aged at room temperature for 135 min. MFI powders are obtained by sedimentation using a centrifuge (Rotina 35, Hettich Lab Technology), flushing with deionized water and subsequent drying of the sediment.

The membrane 2,21,22 is made by placing up to four 16.3 mm×0.5 mm porous and polished alumina disks as support 43 with the polished surface upwards in a 250 mL Teflon beaker. Such support 43 is made from alumina powder (CT 3000 SG, Almatis) that is pelletized at 30 kN hydraulic pressure (GS15011, Specac) and sintered for 30 h at 1150° C. in a furnace (Type 48000, Barnstead Thermolyne). The MFI synthesis solution is added and the Teflon beaker sealed in a stainless steel autoclave and heated for 8 h at 185° C. After rapidly cooling the autoclave down with tap water, the membranes are removed, washed with deionized water and stored overnight in a water bath at 50° C. Drying of the membranes is carried out at 50° C. for at least 3 days in an oven (KB53, Binder). The TPA structuring template is removed by heating the membrane 2,21,22 and powders to 450° C. for 6 h with heating and cooling rates of 30° C. h⁻¹. All experiments are conducted with template-free membranes and powders.

Membrane Characterization

The micropore size distribution of the zeolite powder is assessed by nitrogen sorption with a 3Flex (Micrometrics Instrument Corporation) in the pressure range of p/p₀=4.5·10⁻⁷−0.047, where p and p₀ represent the partial vapour pressure and saturated vapour pressure of the adsorbate gas, respectively of the complex gas mixture. The data is analyzed by the Horwath-Kawazoe method, that assumes cylindrical pore shape, which is consistent with the shape of MFI zeolite membrane 2,21,22. Prior to the analysis, the membrane 2,21,22 was activated (degassed) overnight at 250° C. The macropore size distribution of the alumina support 43 is determined from full nitrogen adsorption and desorption isotherms with a TriStar 3000 (Micromeritics Instrument Corporation) in the pressure range of p/p₀=0.05-0.99. The data is analyzed according to the Barrett-Joyner-Halenda method. The membrane 2,21,22 is degassed under vacuum for 1 h at 150° C. prior to analysis. The film morphology of the membrane and sensing film is investigated by scanning electron microscopy (S-4800, Hitachi) operated at 3 kV.

Gas Detection

The chemoresistive gas detector 3 comprises flame-made (1 mol %) Pd-doped SnO₂ nanoparticles directly deposited onto silicon wafer-based microsubstrates. The detector 3 is either combined with a membrane 2,21,22 or installed alone (for reference tests without membrane 2,21,22) in a sensing cavity 41 comprising stainless steel.

The gas mixing set-up for producing a complex gas mixture 6 is described for example in Sens. Actuators B 2016, 223, 266-273. The complex gas mixture 6 flow is 600 ml min⁻¹ with synthetic air (PanGas 5.0, CₙHₘ and NOₓ≤100 ppb) as carrier gas that is humidified with a water bubbler to achieve 50% relative humidity (RH) forming the sensor baseline. The target compound 64, also called analyte gas, can be formaldehyde (FA) (10 ppm in N₂, PanGas 5.0), acetone (10 ppm in syn. air, PanGas 5.0), ethanol (10 ppm in syn. air, PanGas 5.0), ammonia (NH₃) (10 ppm in syn. air, PanGas 5.0), isoprene (10 ppm in syn. air, PanGas 5.0). For tests with TIPB (1,3,5-Triisopropylbenzene 95%, Sigma-Aldrich), the complex gas mixture 6 gas is obtained as follows: 5 ml of liquid TIPB is poured into a 50 mL wide neck flask. TIPB vapor is formed in its headspace that is purged with 100 mL min⁻¹ of synthetic air. That way, ˜18 ppm of TIPB in synthetic air/complex gas mixture 6 is obtained, as measured with a proton transfer reaction time-of-flight mass spectrometer (PTR-TOF-MS1000, Ionicon).

The detector 3 response S is defined as:

$$S = \frac{{R}_{air}}{{R}_{analyte}} - 1$$

where Rₐᵢᵣ and R_{analyte} represent the film resistances in absence and presence of the analyte, respectively. Response and recovery times are defined as the time needed to reach and recover 90% of the response resistance change, respectively. Signal-to-noise ratio (SNR) is defined as the ratio of the signal ΔR=Rₐᵢᵣ−R_{analyte} to the standard deviation of the noise.

Membrane 2,21,22—Detector 3 System for Detection of a Target Compound 64 (Gas Analyser 1)

The gas analyser 1, as membrane 2,21,22—detector 3 system, is illustrated schematically in FIG. 1a. The complex gas mixture 6, also called real gas mixture, (e.g. indoor air (Indoor Air 1994, 4 (2), 123-134) or breath (J. Breath Res. 2014, 8 (1), 014001)) comprises a large number of different molecules respectively compounds. These can be separated respectively simplified with a membrane 2,21,22, for example a zeolitic membrane 2 as described above. Ideally, only a single target compound 64 (medium sized ball) can permeate respectively traverse through the membrane 2,21,22 while other interfering compounds of the complex gas mixture 6 (small and big balls) are held back. Eventually, the target compound 64 is detected with a chemoresistive detector 3 placed after the membrane 2,21,22 in a sensing cavity 41 and the concentration of the target compound 64 is deduced from the detector's 3 electrical resistance change (i.e. response). The outer wall of the sensing cavity 41 is not shown.

The membrane 2,21,22 is coin-type and rather small (d=16.3 mm) and comprises a compact and coherently grown ˜3 μm MFI membrane 2,21,22 that is supported on a porous Al₂O₃ support 43. The pore diameter distribution for the MFI membrane 2,21,22 (left side; dₚ<1 nm) and α-Al₂O₃ support 43 (right side; dₚ>10 nm) are shown in FIG. 1b: MFI micropores were measured to be in a size range of 0.57 to 0.61 nm, slightly larger than silicalite (alumina-free MFI—Nature 1978, 271, 512-516). In contrast, the α-Al₂O₃ support pellet 43 has pores>40 nm, significantly larger than most target compounds 64. FIG. 1d depicts the pore diameter dₚ in nm (nano meter) as a function of the normalized pore volume V for the MFI membrane 2 and the Al₂O₃ support element 43.

The applied detector 3 is a chemo-resistive type and consists of a semiconductive metal-oxide film on Si wafer-based micodetector 3 substrates with a size smaller than a match head. In particular, the sensing film is formed by flame-made Pd-doped SₙO2 nanoparticles that aggregate to a highly porous network providing high surface area available for target compound 64 interaction. Such flame-made and nanostructured Pd-doped SnO2 sensors (without membrane) are highly sensitive and can detect, for instance, formaldehyde FA down to 3 ppb (at 90% RH) with typical response times of ˜2 min and always complete recovery during continuous application (ACS Sens. 2016, 1 (5), 528-535).

The sensing cavity 41, membrane 2,21,22 and detector 3 are decoupled (mechanically and thermally) and thus can be combined flexibly and operated independently. Due to their compact and modular design, such membrane 2,21,22—detector 3 systems (analyser 1) can be easily incorporated into analysers, e.g. compact indoor air monitors or portable breath analyzers, and they feature low sensor power consumption of ˜500 mW at 400° C.

MFI/Al2O3 Membrane 2,21,22 Turns Detector 3 Formaldehyde-Selective

To evaluate the membrane effect on the detector 3, various target compounds 64 are tested covering a wide range of physical and chemical properties. FIG. 2 lists the chosen target compounds 64 with their kinetic diameter (kd) in comparison to the determined MFI pore size (ps). 1,3,5-TIPB (TIPB—diagonal shaded) represent a symmetric molecule being larger than the pore size of the MFI-membrane 2 (shown as diamond outlined pattern) and that way, the size filtering effect of the membrane can be assessed. Formaldehyde (FA—vertical lines), isoprene (Isop—large squared), acetone (Ac—small squared), ethanol (EtOH—diagonal lines) and ammonia (NH₃—horizontal lines) are smaller than the membrane 2 pores and they are selected due to their different functional groups, introducing a diversity of chemical properties, and their relevance for indoor air monitoring (Indoor Air 1994, 4, 123-134) and breath analysis (Breath Res. 2014, 8, 014001).

FIG. 3a shows the change in detector 3 resistance (R) of the chemoresistive Pd-doped (1 mol %) SnO2 detector 3 without membrane upon exposure to 1 ppm of acetone (dotted line—Ac), Ammonia (dash dotted line—NH₃), ethanol (long dash line—EtOH), isoprene (dashed line—Isop) and formaldehyde (solid line—FA) at 400° C. and 50% RH. When injecting FA, the detector 3 resistance drops rapidly from 128 to 10.2 kΩ, corresponding to a response of 11.3. Also the other target compounds 64 are detected clearly and the corresponding responses (normalized to maximum response of Formaldehyde (FA)—Sₙ—normalized response) are shown in FIG. 3b. This indicates the rather non-specific character of Pd-doped SnO₂ and thus it cannot detect formaldehyde (FA) selectively in a complex gas mixture 6 as it is not possible to distinguish it from interfering gases (e.g. acetone).

This problem is solved when adding the membrane 2,21,22. Indeed, when exposing the detector 3 now to the different target compounds 64, only formaldehyde (FA) is detected (FIG. 3c, and FIG. 3d), so the membrane 2,21,22 turns the non-specific Pd-doped SnO2 detector 3 formaldehyde-selective. Thus the detector 3 does hardly respond to other gases. More specific, the FA selectivity to acetone is improved to >100 and the one to NH3, isoprene, ethanol and TIPB is even >1000, much higher than without membrane 2,21,22. So it seems that FA can permeate through the MFI/Al2O3, similar as observed before (J. Membr. Sci. 2004, 240 (1), 159-166), while other compounds are held back by the membrane. Actually, TIPB should be separated due to its larger molecular size compared to the distinct MFI pore diameter range (FIG. 2). This size cut-off is rather important for indoor air monitoring and breath analysis since both gas mixtures contain a myriad of such larger molecules potentially interfering with the sensor. The MFI layer should filter out all of them similarly efficient as TIPB. In case of other compounds smaller than the size cut-off (i.e. isoprene, NH₃, acetone and ethanol), these might be separated due to their different sorption and diffusion properties.

Size cut-off for TIPB represents many other unknown gases with larger size than the pore size.

The calculated selectivities (S_{FA}/S_{target}) for the Pd-doped SnO2 detector 3 with and without membrane are shown in Table 1 along with other state-of-the-art FA gas detectors: while the Pd-doped SnO2 detector 3 features rather weak selectivity, this is dramatically improved with membrane 2,21,22. Actually, the selectivity to acetone is >100 while the one to NH3, isoprene and ethanol is even >1000. This is also superior to other metal-oxide sensors, such as Ag-doped LaFeO3 (J. Mater. Chem. C 2014, 2 (47), 10067-10072) or TiO2 nanotubes (Sens. Actuators B 2011, 156 (2), 505-509) that had been proposed as FA sensors.

In Table 1, the FA selectivity of the membrane-sensor system (analyser 1) is benchmarked with other state-of-the-art FA gas detectors. Various chemoresistive gas detectors, including metal-oxides (e.g. Ag-doped LaFeO₃—J. Mater. Chem. C 2014, 2 (47), TiO₂ nanotubes—Sens. Actuators B 2011, 156 (2), 505-509) and metal-organic frameworks (e.g ZIF-67—Inorg. Chem. 2014, 53 (11), 5411-5413) had been developed for high FA selectivity. However, all are outperformed clearly by the present membrane-sensor system 1. While this indicates the immediate impact of the novel membrane approach, it shows also the limitations of sensor material optimization.

TABLE 1
Selectivity of formaldehyde detectors

| | | | Formaldehyde selectivity S_{Formaldehyde}/Sₓ [—] | | | |
|---|---|---|---|---|---|---|
| | Type | Material | Ammonia | Ethanol | Acetone | Isoprene |
| chemo-resistive | MOx sensor + | Pd:SnO₂ + | >1000 | >1000 | >100 | >1000 |
| | membrane | MFI/Al₂O₃ | | | | |
| | MOx sensor only | Pd:SnO₂ | 12 | 3 | 26 | 1.4 |
| | | Ag:LaFeO₃ | 35 | 27 | 50 | — |
| | | TiO₂ nanotubes | 12 | 57^{a} | — | — |
| | ZIF sensor | ZIF-67 | 43 | 2^{b} | 2 | — |
| | Coated sensor | ZIF-8 coated ZnO | 5 | 7 | 11 | — |
| optical | Photoelectric | Colorimetric sensor | — | high | 8 | — |
| | photometry | | | | | |
| | Fiber-optic | NADH based flow cell | — | high | high | — |

^{a}linearly interpolated to same concentrations;
^{b}selectivity to methanol

In another study, zeolitic ZIF-8 had been applied directly as coating onto ZnO to pre-filter molecules (ACS Sens. 2016, 1 (3), 243-250), similar to the membrane here. While improved FA selectivity was observed, a distinct size cut-off was not obtained (ACS Sens. 2016, 1 (3), 243-250). In fact, larger molecules than the ZIF-8 pore size (0.34 nm), e.g. ethanol and acetone, were detected by the sensor (ACS Sens. 2016, 1 (3), 243-250) resulting in significantly lower FA selectivity compared to the membrane-sensor (analyser 1) configuration here (Table 1). This is probably associated to the high temperature of the coating (300° C.) that is thermally coupled to the ZnO and thus heated as well. This could lead to catalytic fragmentation of larger molecules on the ZIF-8 surface and that way, smaller product molecules can enter the ZIF-8 pores undermining the desired size cut-off. So the thermal decoupling of the present membrane-sensor approach is rather important allowing independent operation of the both to avoid such unwanted effects.

Finally, FA as a target compound 64 can be detected also by optical sensors and these achieve rather high selectivity with respect to ethanol (Table 1) while their performance for NH3 and isoprene is unknown (FP-30 RKI Instruments, Biosens. Bioelectron. 2010, 26 (2), 854-858). However, while the commercial FA detector FP-30 (RKI Instruments) features insufficient selectivtiy to acetone (Biosens. Bioelectron. 2010, 26 (2), 854-858), fiber-optic sensors that require enzymes for irreversible reaction with FA (Biosens. Bioelectron. 2010, 26 (2), 854-858) might have rather limited life-time. In case of the later, these enzymes are continuously consumed and depleted at some point, as observed for similar devices with a performance deterioration of 80% after 6 days (Anal. Chem. 1994, 66 (20), 3297-3302).

Response and Recovery Times

Fast response and recovery times are desirable properties for gas sensors. While the Pd-doped SnO2 detector 3 without membrane 2,21,22 possesses response and recovery times of 1 and 9 min, respectively, introducing the membrane delays them to 8 and 72 min. An increase is expected from gas diffusion theory, as the membrane 2,21,22 and, in particular, its dense and microporous MFI layer represents an additional permeation barrier. However, this could be minimized by reducing the MFI-membrane 2,21,22 layer thickness. In principle, maximal molecular diffusion would be obtained when the layer thicknesses approaches the dimensions of a zeolite's single unit cell. Previous studies had demonstrated the synthesis of 2 nm thick MFI nanosheets (Nature 2009, 461, 246-249), much thinner than the 3 μm MFI layer applied here.

Lower Limit of Detection and Calibration Curve

The detection of formaldehyde FA levels below 100 ppb is crucial for indoor air monitoring to distinguish normal from hazardous conditions (Crit. Rev. Toxicol. 2011, 41 (8), 672-721) and also in medical diagnostics where typical breath formaldehyde (FA) concentrations in lung cancer patients and healthy humans are smaller (Int. J. Cancer 2010, 126 (11), 2663-2670). FIG. 5 shows the detector 3 resistance [R (kΩ)] change over time [t (min)] to 100, 70, 60 and 30 ppb of formaldehyde FA with the membrane 2,21,22 at 50% RH and 400° C. When exposed to 100 ppb, the resistance R drops rapidly from 35.1 to 25 kΩ corresponding to a response of 0.4 within a response time of <14 min. Most notably, formaldehyde FA levels down to 30 ppb can be detected with a high signal-to-noise ratio (SNR, >80), which is sufficient for breath analysis and indoor air monitoring. Actually, the extrapolated lower limit of detection (LOD) for formaldehyde FA is 0.2 ppb at a SNR=1. This is rather comparable to single Pd-doped SnO₂ (0.1 ppb at 90% RH—ACS Sens. 2016, 1 (5), 528-535) indicating the little interference of the membrane 2,21,22, and superior to zeolite-coated ZnO (5.6 ppm 50-60% RH—ACS Sens. 2016, 1 (3), 243-250). Additionally, the membrane 2,21,22—detector 3 system (analyser 1) has excellent formaldehyde (FA) resolution, in fact, the responses for 60 to 70 ppb are clearly distinguishable.

Another important feature for a gas detector 3 is repeatable usability. As shown in FIG. 5, the membrane 2,21,22—detector 3 system (analyser 1) always fully recovers the initial baseline when flushed with air enabling repeatable exposure to formaldehyde (FA). This indicates reversible and fast FA permeation through the membrane 2,21,22 and interaction with the detecting structure without any observable deactivation. These detector responses are also stable and nicely reproducible. In fact, when exposing the detector 3 twice to 60 ppb of formaldehyde (FA), the same response resistance is achieved (dashed line, FIG. 5). Reproducible responses are also obtained during continuous operation for several days (results not shown here) without any observable degradation. Similar flame-made Pt-doped SnO₂ had shown stable detector 3 performance during 20 days of continuous operation at 10% RH (J. Nanopart. Res. 2006, 8 (6), 783-796) and also MFI membranes 2,21,22 maintained constant selectivity and permeance for at least 5 days even with concentrated gas mixtures (Microporous Mesoporous Mater. 2014, 192, 76-81). While these first results are promising, long-term stability still needs to be investigated.

The detector 3 calibration curve (at 400° C.) for formaldehyde (FA—64) in the range of 0-1000 ppb at 50% RH is shown in FIG. 4 (triangles). FIG. 4 depicts the detector 3 calibration curve for formaldehyde in the range of 0-1000 ppb at 400° C. and 50% RH (triangles). FIG. 4 illustrates the detector 3 response S versus the formaldehyde (FA) concentration c (ppb). Interestingly, this calibration curve does not change even when introducing 1 ppm of NH₃, acetone, isoprene and EtOH, all at the same time (squares) highlighting the excellent formaldehyde selectivity of the membrane 2,21,22—detector 3 system (gas analyser 1). That way, hazardous FA levels above the recommended indoor air limit (100 ppb—A—Crit. Rev. Toxicol. 2011, 41 (8), 672-721) and eye irritation threshold (500 ppb—B—Regul. Toxicol. Pharmacol. 2008, 50 (1), 23-36) can be detected rapidly. The responses increase continuously with increasing FA concentration and this allows to distinguish them clearly. That way, and most importantly, FA levels exceeding the recommended indoor air limit of 100 ppb (Crit. Rev. Toxicol. 2011, 41 (8), 672-721) and the eye irritation threshold at 500 ppb (Regul. Toxicol. Pharmacol. 2008, 50 (1), 23-36) can be rapidly recognized to protect from potential cancer risks and sensory impairment.

Selectivity in Gas Mixtures

Indoor air and breath consist of complex gas mixtures 6 and some target compounds 64 may be present at even higher concentrations than formaldehyde (FA). FIG. 4 (squares) shows the detector 3 calibration curve to formaldehyde (FA) when additional 1 ppm of NH₃, acetone, isoprene and ethanol are introduced at the same time. Remarkably, the calibration curve for FA (even at 30 ppb) does not change despite the four interfering compounds at substantially higher concentrations. This emphasizes the excellent separation properties of the membrane 2,21,22 being superior to E-noses that can trace FA in comparable gas mixtures as well, however, with an estimation error of ˜9 ppb (ACS Sens. 2016, 1, 528-535).

Further embodiments are evident from the dependent patent claims. Features of the method claims may be combined with features of the device claims and vice versa.

While the invention has been described in present preferred embodiments of the invention, it is distinctly understood that the invention is not limited thereto, but may be otherwise variously embodied and practised within the scope of the claims.

FIG. 6 shows a schematic sketch of an analyser 1 with a sensing cavity 41, a membrane 2,21,22 with support element 23 and a detector 3 within the sensing cavity 41. The analyser 1 further comprises an inlet 51 and an outlet 52. The separate membrane 2,21,22 is closing the cavity 41. A transition between the membrane 2,21,22 and the cavity 41 is sealed, by an O-ring,

1. A gas analyzer for detection of a target compound in a complex gas mixture in the gas phase,
the analyzer comprising
a detector for detecting the target compound and
a sensing cavity,
wherein the detector is arranged in the cavity;
the analyzer further comprises a first membrane being separate,
wherein the first membrane is equipped to close the sensing cavity, and
wherein the first membrane is equipped for simplifying the composition of the complex gas mixture into a first gas mixture,
wherein said first membrane is capable of performing a chemical and a physical separation of composition of the complex gas mixture,
wherein the first gas mixture comprises the first target compound and
wherein the first membrane is equipped to let the first gas mixture traverse through the first membrane into the sensing cavity;
wherein the detector is equipped to detect the first target compound.

2. The analyzer according to claim 1, wherein the first membrane comprises a pore size with a diameter of maximally 10 nm.

3. The analyzer according to claim 1, comprising an inlet and an outlet, wherein the inlet is arranged such that the complex gas mixture is guided onto the membrane closing the sensing cavity, and the first membrane is arrangeable between the inlet and the outlet, and wherein the outlet is arranged in an axial and/or non-axial orientation with respect to the inlet.

4. The analyzer according to claim 1, wherein the membrane is equipped to block the direct flow of the complex gas mixture into the sensing cavity.

5. The analyzer according to claim 1, wherein
the sensing cavity comprises a detector housing with an opening, wherein the first membrane is arrangeable in the opening and/or
the sensing cavity comprises a base part, wherein the first membrane is fastenable to the base part and builds the sensing cavity together with the base part in a fastened state.

6. The analyzer according to claim 1, further comprising a separate second membrane, wherein the first membrane is interchangeable with the second membrane,
wherein the second membrane is equipped to close the sensing cavity, and
wherein the second membrane is equipped for simplifying the composition of the gas mixture into a second gas mixture,
wherein the second gas mixture comprises a second target compound and
wherein the second membrane is equipped to let the second gas mixture traverse through the second membrane into the sensing cavity;
wherein the detector is equipped to detect the concentration of the second target compound.

7. The analyzer according to claim 6, further comprising at least a first sensing cavity and a second sensing cavity,
wherein the first membrane is equipped to close first sensing cavity and wherein the second membrane is equipped to close the second sensing cavity,
wherein in the first sensing cavity a first detector is arranged and the first detector is equipped for detecting a first target compound and
wherein in the second sensing cavity a second detector is arranged, wherein the second detector is equipped for detecting a second target compound.

8. The analyzer according to claim 7, wherein the second sensing cavity is arranged in a parallel manner and/or in a serial manner with the first sensing cavity.

9. The analyzer according to claim 6, wherein the first membrane and the second membrane are equipped to close the sensing cavity.

10. The analyzer according to claim 1, wherein the analyzer is equipped for selective detection of the concentration of the target compound in the complex gas mixture and wherein the detector is equipped for detecting the concentration of the target compound.

11. The analyzer according to claim 1, wherein the analyzer is equipped for detection of target compound in the gas phase at a trace level.

12. The analyzer according to claim 1, wherein the first membrane and/or the second membrane is closing the sensing cavity, wherein a transition between the membrane and the sensing cavity is sealed.

13. The analyzer according to claim 1, wherein the first membrane respectively the second membrane comprises a microporous material, with a pore size comparable to the size of the target compound.

14. The analyzer according to claim 1, wherein the detector is at least one of a chemoresistive detector, a mass spectrometer, and an optical system, and wherein the detector is selected from a group comprising:
a doped SnO2 detector, in particular doped with Pt, Pd, Si, Ti;
a WO3 detector, in particular a Si-doped WO3 detector, in particular a epsilon phase of Si-doped WO3 detector;
a MoO3 detector, in particular a Si-doped MoO3 detector, in particular an alpha phase of Si-doped MoO3 detector;
a ZnO detector, in particular a Ti-doped ZnO;
a CuBr detector and
a CuO—SnO2 heterojunction.

15. The analyzer according to claim 1, wherein the target compound is a sulfuric compound, a ketone, a hydrocarbon, an aldehyde, an pnictogen hydride, an acid, an alcohol, and/or hydrogen.

16. The analyzer according to claim 1, wherein said analyzer is equipped
for use in medical and/or biological fluid analysis;
for use in breath analysis, in particular in lung cancer detection from exhaled breath; and/or
for use in analysis of skin emissions, and/or
for use in headspace analysis of fluids; and or for use in air quality analysis, in particular air quality monitoring of target compound released from indoor sources; and/or
for use in food quality assessment, food processing control and/or monitoring; and/or
for use in monitoring and/or controlling agricultural and/or chemical processes and products; and/or
for use in environmental analysis or monitoring and/or controlling exhaust emission; and/or
for use in detection of explosives and other hazardous compounds.

17. Use of the analyzer according to claim 1 for medical and/or biological fluid analysis; for breath analysis; for analysis of skin emissions; for headspace analysis of fluids; for air quality analysis; for food processing control; for food quality assessment; for air quality analysis, for indoor air analysis; for monitoring and/or controlling agricultural and/or chemical processes and products; and/or for environmental analysis; monitoring and/or controlling exhaust emission; and/or for detection of explosives and other hazardous compounds.

18. A method for detecting a target compound in a complex gas mixture comprising the step of:
providing an analyzer according to claim 1;
letting a complex gas mixture interact with the first mixture, respectively, a second membrane,
wherein the first gas mixture traverses through the membrane into the sensing cavity; and
wherein the detector detects the target compound.

19. The analyzer according to claim 1, wherein the first membrane comprises a pore size with a maximum diameter of 2 nm.

20. The analyzer according to claim 1, wherein the first membrane comprises a pore size with a maximum diameter of 1 nm.
